(19) United States
(12) Patent Application Publication
(10) Pub. No.: US 2022/0244216 A1
(43) Pub. Date: Aug. 4, 2022

(54) BIOLOGICAL CHIP TESTING SYSTEM

(71) Applicant: HERMES TESTING SOLUTIONS INC., Hsinchu City (TW)

(72) Inventor: Chi-Ming Yang, Hsinchu City (TW)

(21) Appl. No.: 17/211,320

(22) Filed: Mar. 24, 2021

(30) Foreign Application Priority Data
Feb. 2, 2021 (TW) 110103902

(51) Int. Cl. G01N 27/414; G01N 33/543

(52) U.S. Cl. CPC G01N 27/4145; G01N 33/54373

(57) ABSTRACT

A biological chip testing system includes a carrier table, a liquid injection device, a probe card and a tester. The carrier table is used to carry a biological chip including at least one biological field effect transistor and a gate conductive contact, a drain conductive contact and a source conductive contact, which are electrically connected to the biological field effect transistor. The liquid injection device supplies a liquid to be tested to a detection area of the biological chip. The probe card includes a plurality of probes which are used to selectively contact the gate conductive contact, the drain conductive contact and the source conductive contact, wherein the probe contacting the gate conductive contact does not contact the liquid. The tester measures the change of the electrical signal of the biological field effect transistor. The above-mentioned biological chip testing system does not require frequent replacement of gate electrodes.

### BACKGROUND OF THE INVENTION

### 1. Field of the Invention

The present invention relates to a chip testing system, particularly to a biological chip testing system.

### 2. Description of the Prior Art

A biochip uses the variation of electric and/or electrochemical characteristics to inspect biological or environmental materials. The biological field-effect transistor (BioFET) is an important element of biochips. While the tested object combines with the gate of the BioFET or the receptor fixed to the gate, the drain current of the BioFET will vary with the gate voltage. The magnitude of variation is dependent on the type and amount of the bonds of the tested object and used to determine the type and/or amount of the tested object.

Similar to general semiconductor chips, biochips should be fully tested to screen out unqualified products. Different from general semiconductor chips, biochips normally test liquid samples. Therefore, an appropriate liquid sample will be filled into the detection area of a biochip to test the biochip. Refer to FIG. 1. In a conventional testing method, a tested liquid L is filled into the detection area of a biochip 100; probes 21 and 22 respectively contact conductive contacts 101 and 102, which are electrically connected with the source and the drain of the BioFET separately; the gate electrode 23 touches the tested liquid L. During test, the gate electrode 23 supplies gate voltage and measures the drain current of the BioFET. The gate electrode 23, which has contacted the tested liquid L many times, may causes test errors. In order to maintain the stability of tests, the gate electrode 23 should be replaced after a given number of cycles of tests. As the gate electrode 23 is normally made of silver or silver chloride, frequent replacement of the gate electrode 23 would impede decreasing test cost.

Accordingly, the manufacturers are eager to develop a biochip testing system to reduce the cost of testing biochips.

### SUMMARY OF THE INVENTION

The present invention provides a biological chip testing system, wherein the biological chip testing is realized by that the probe, which supplies a voltage to the gate, contacts the conductive contact electrically connected with the gate structure of the BioFET, whereby to prevent the probe from contacting the tested liquid and exempt the biological chip testing system from frequent replacement of the gate electrodes, wherefore biological chip testing cost is reduced.

In one embodiment, the biological chip testing system of the present invention comprises a carrier table, a liquid injection device, a probe card and a tester. The carrier table is used to carry a biological chip or a wafer containing at least one biological chip. The biological chip includes at least one detection area and a conductive contact area. Each detection area includes at least one biological field-effect transistor (BioFET). The conductive contact area includes a gate conductive contact, a drain conductive contact, and a source conductive contact, which are electrically connected with the BioFET. The liquid injection device supplies a tested liquid to the detection area and removes the tested liquid from the detection area. The probe card includes a plurality of probes. The probes selectively contact the gate conductive contact, the drain conductive contact and the source conductive contact inside the conductive contact area, wherein the probe contacting the gate conductive contact does not contact the tested liquid. The tester is electrically connected with the probe card, supplies a voltage to the BioFET and measures the variation of an electrical signal of the BioFET.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a diagram schematically showing a conventional biological chip testing method;

FIG. 2 is a diagram schematically showing a biological chip testing system according to one embodiment of the present invention;

FIG. 3 is a top view schematically showing a biological chip; and

FIG. 4 is a diagram schematically showing a biological chip testing system according to another embodiment of the present invention.

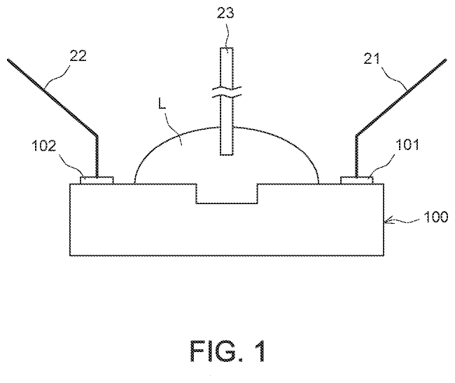

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

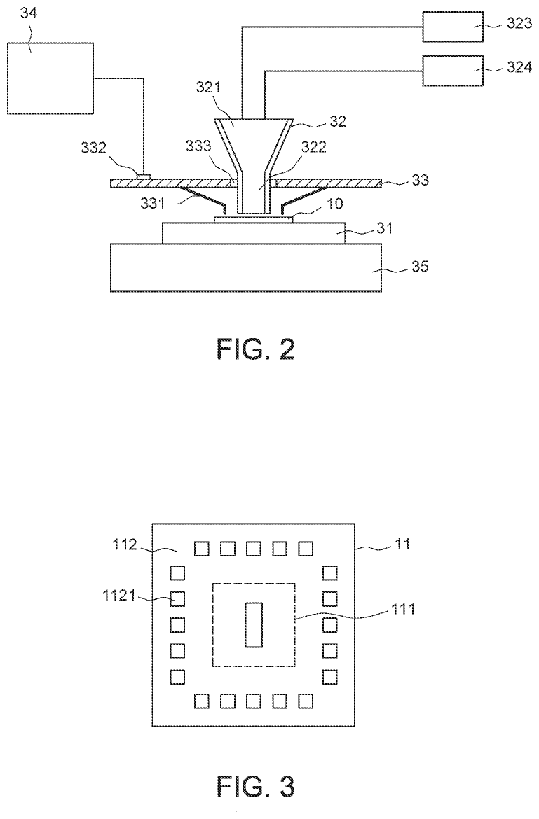

Refer to FIG. 2 and FIG. 3. In one embodiment, the biological chip testing system of the present invention comprises a carrier table 31, a liquid injection device 32, a probe card 33 and a tester 34. The carrier table 31 is used to carry a biological chip 11 or a wafer 10 containing at least one biological chip 11. The biological chip 11 includes at least one detection area 111 and a conductive contact area 112. Each detection area 111 includes at least one biological field-effect transistor (BioFET). The BioFET includes a gate structure, a drain and a source. A plurality of conductive contacts 1121 is arranged on the conductive contact area 112, including a gate conductive contact electrically connected with the gate structure of the BioFET, a drain conductive contact electrically connected with the drain area of the BioFET, and a source conductive contact electrically connected with the source area of the BioFET. It is easily understood: the gate structure of the BioFET may be appropriately processed according to the tested object. For example, the biological chip may be applied to nucleic acid hybridization tests, polymerase activity tests, nucleic acid and protein sequencing, immunological tests (such as the enzyme-linked immunosorbent assay (ELISA)), bisulfite methylation tests for gene methylation modes, protein tests, protein binding tests (such as the tests for protein-protein binding, protein-nucleic acid binding, and protein-ligand binding), enzyme tests, enzyme coupling tests, screening for enzyme inhibitors and enzyme activators, etc. However, the biological chip is not limited to the abovementioned applications in the present invention.

The liquid injection device 32 is used to supply a tested liquid to the detection area 111 and remove the tested liquid from the detection area 111. In one embodiment, the liquid injection device 32 includes a reservoir 321, a first liquid supply unit 323, and a liquid drainage unit 324. The first liquid supply unit 323 is connected with the reservoir 321 and supplies a tested liquid to the reservoir 321. The reservoir 321 has at least one liquid delivering orifice 322. During operation, the liquid delivering orifice 321 may be pressed against the detection area 111 of the biological chip 11, whereby the tested liquid stored in the reservoir 321 can exactly contact the detection area 111 and would not overflow to the conductive contact area 112. The liquid drainage unit 324 is also connected with the reservoir 321. After the test is completed, the liquid drainage unit 324 may draw out the liquid inside the reservoir 321.

The probe card 33 includes a plurality of probes 331. The probes 331 selectively contact the corresponding gate conductive contact, the corresponding drain conductive contact and the corresponding source conductive contact to facilitate the tester 34 to undertake tests. In one embodiment, the probe card 33 includes a hole 333 corresponding to the liquid delivering orifice 322 of the reservoir 321. The liquid delivering orifice 322 passes through the probe card 33 via the hole 333 to press against the detection area 111 of the biological chip 11.

The tester 34 is electrically connected with the probe card 33. For example, the tester 34 is electrically connected with the probe card 33 via a D-type connection port or another appropriate connection port 332. Through the plurality of probes 331 of the probe card 33, the tester 34 supplies a voltage to the BioFET inside the biological chip and measures the variation of an electrical signal of the BioFET. For example, the tester 34 supplies a gate voltage to the gate of the BioFET; while the tested object combines with the gate of the BioFET or the receptor fixed to the gate, the drain current of the BioFET will vary with the change of the characteristics of the channel of the BioFET; the tester 34 measures the variation of the drain current to determine whether the biological chip is qualified. Alternatively, the tester 34 provides a bias to the source and drain of the BioFET and measures the gate voltage in the channel conversion of the BioFET to determine whether the biological chip is qualified.

The gate conductive contact of the BioFET is disposed inside the conductive contact area 112 of the biological chip 11. The tested liquid would not overflow to the conductive contact area 112. Therefore, the probe, which contacts the gate conductive contact, will not contact the tested liquid during test. Thereby, the stability of tests can be promoted, and the gate electrode needn't be replaced frequently.

Refer to FIG. 2. In one embodiment, the biological chip testing system further comprises an elevation device 35. The elevation device 35 adjusts the altitude of the carrier table 31 to modify the relative position of the carrier table 31 and the probe card 33, whereby to make the probes 331 of the probe card 33 contact the plurality of conductive contacts 1121 of the biological chip 11 for testing the biological chip or make the probes 331 separate from the biological chip 11 for replacing the tested chip. In the embodiment shown in FIG. 2, the elevation device 35 is coupled to the carrier so as to adjust the distance between the carrier table 31 and the probe card 33. However, the present invention is not limited by this embodiment. In one embodiment, the elevation device 35 is coupled to the probe card 33 and controls the altitude of the probe card 33 to adjust the distance between the carrier table 31 and the probe card 33.

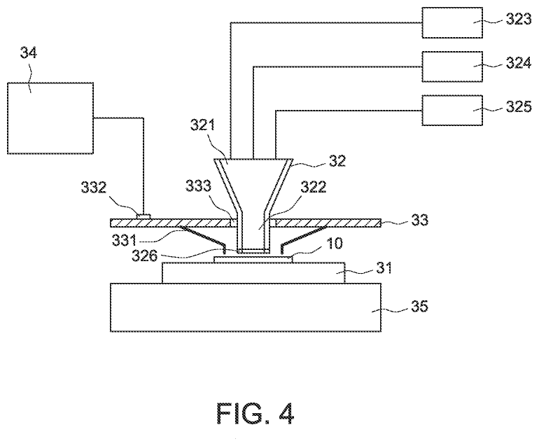

Refer to FIG. 4 for a biological chip testing system according to another embodiment of the present invention. The elements in FIG. 4, whose functions are the same as the functions of the elements in FIG. 2, are designated with the same symbols (numerals), and the functions thereof will not repeat herein. In comparison with the embodiment shown in FIG. 2, the liquid injection device 32 of the embodiment shown in FIG. 4 further includes a second liquid supply unit 325. The second liquid supply unit 325 is connected with the reservoir 321 and supplies a cleaning liquid to the reservoir 321 to clean the detection area 111 of the biological chip 11 after tests. Alternatively, the second liquid supply unit 325 supplies a cleaning liquid to clean the reservoir 321 before tests.

Refer to FIG. 4 again. In one embodiment, the liquid injection device 32 further includes a buffering element 326, which is disposed at the end of the liquid delivering orifice 322. While the liquid delivering orifice 322 of the reservoir 321 is pressed against the detection area 111 of the biological chip 11, the buffering element 326 may prevent the liquid delivering orifice 322 from damaging the biological chip 11. Further, the buffering element 326 may increase the airtightness between the liquid delivering orifice 322 and the biological chip 11 lest the tested liquid overflow out of the detection area 111.

In the biological chip testing system of the present invention, the probe of the probe card contacts the conductive contact of the gate structure, which is electrically connected with the BioFET, to supply voltage for tests. Therefore, the present invention is characterized in exempting the probes from contacting the tested liquid. Hence, the present invention can avoid test errors resulting from the contact of the gate electrode and the tested liquid. Further, the present invention can decrease the number of replacing gate electrodes and thus can reduce the testing cost.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. A biological chip testing system, comprising:
a carrier table, used to carry a biological chip or a wafer including at least one biological chip, wherein the biological chip includes at least one detection area and a conductive contact area; each detection area includes at least one biological field-effect transistor (BioFET); the conductive contact area includes a gate conductive contact, a drain conductive contact, and a source conductive contact, which are electrically connected with the BioFET;
a liquid injection device, supplying a tested liquid to the detection area of the biological chip and removing the tested liquid from the detection area;
a probe card, including a plurality of probes, wherein the probes selectively contact the gate conductive contact, the drain conductive contact and the source conductive contact, and wherein the probe contacting the gate conductive contact does not contact the tested liquid; and
a tester, electrically connected with the probe card, supplying a voltage to the BioFET, and measuring variation of an electrical signal of the BioFET.

2. The biological chip testing system according to claim 1 further comprising:
an elevation device, coupled to at least one of the carrier table and the probe card, and adjusting a relative position of the carrier table and the probe card to make the plurality of probes selectively contact the gate conductive contact, the drain conductive contact, and the source conductive contact.

3. The biological chip testing system according to claim 1, wherein the liquid injection device includes:
a reservoir, storing the tested liquid, and having at least one liquid delivering orifice, wherein the liquid delivering orifice is configured to selectively press against the detection area of the biological chip to make the tested liquid contact the detection area;
a first liquid supply unit, connected with the reservoir, and used to supply the tested liquid to the reservoir; and
a liquid drainage unit, connected with the reservoir, and used to draw out a liquid inside the reservoir.

4. The biological chip testing system according to claim 3, wherein the liquid injection device further includes:
a second liquid supply unit, connected with the reservoir, and used to supply cleaning liquid to the reservoir.

5. The biological chip testing system according to claim 3, wherein the liquid injection device further includes:
a buffering element, disposed at one end of the liquid delivering orifice, and used to press against the detection area of the biological chip.
